(19) United States
(12) Patent Application Publication
(10) Pub. No.: US 2022/0192554 A1
(43) Pub. Date: Jun. 23, 2022

(54) PUSH-TYPE SAFETY PROTECTION MECHANISM OF BLOOD COLLECTION NEEDLE

(71) Applicant: Shanghai Baoshun Medical Apparatus Co., Ltd., Shanghai (CN)

(72) Inventors: Shaojun ZOU, Shanghai (CN); Xiaolong ZOU, Shanghai (CN)

(73) Assignee: Shanghai Baoshun Medical Apparatus Co., Ltd., Shanghai (CN)

(21) Appl. No.: 17/128,125

(22) Filed: Dec. 20, 2020

(51) Int. Cl. A61B 5/15

(52) U.S. Cl. CPC A61B 5/150534; A61B 5/150435

(57) ABSTRACT

The present invention discloses a push-type safety protection mechanism of a blood collection needle. The push-type safety protection mechanism comprises a sleeve member and a valve core member, wherein the valve core member is provided with three parts, namely a needle seat part, a sleeving part and a stopping part; a plurality of limiting grooves is formed in the stopping part; the sleeve member is provided with two parts, namely a first sleeve part positioned at a front section and a second sleeve part positioned at a rear section; an elastic button higher than the side wall of the first sleeve part is arranged on the side wall of the valve core member; a limiting opening matched with the elastic button is formed in the side wall of the first sleeve part; a limiting sliding cavity for accommodating the elastic button and the valve core member is formed from a position of the first sleeve part from the limiting opening to the inner side of the second sleeve part, and the limiting sliding cavity guides and limits front-back movements of the elastic button and the valve core member; and a plurality of elastic limiting protrusions matched with the limiting grooves is arranged on the inner wall of the tail sliding cavity of the second sleeve part. Through the design of the elastic button, medical staff can operate the blood collection needle with the left hand or the right hand alone during use.

### TECHNICAL FIELD

The present invention relates to a push-type safety protection mechanism of a blood collection needle, and belongs to the technical field of medical safety.

### BACKGROUND

In clinical work, the occurrence of injection-related needle-stick injuries are random and uncontrollable, and thereby nurses are a high-risk group suffering from needle-stick injuries. The hazards of unsafe injections to society include others being stabbed by used needles and improper handling of used injection apparatuses, which leads to the spread of infections in society. Standard prevention was proposed by the U.S. Centers for Disease Control and Prevention in 1995 and implemented throughout the United States in 1996. It was introduced by China in 1999 and incorporated into the “Hospital Infection Management Regulations (Trial)” issued by the Ministry of Health in 2000. The viewpoint of the standard prevention expressed is that: since all patients' blood, body fluids, excreta, and secretions may be infectious, all patients must be isolated regardless of whether there is obvious blood contamination or contact with incomplete skin and mucous membranes. Therefore, the large-scale use of disposable blood collection needles will inevitably bring about the problem in safe disposal of the disposable blood collection needles.

A safety blood collection needle is generally used at present. A vacuum blood collector is composed of a vacuum blood collection tube, a blood collection needle, a connecting tube and a needle holder. The vacuum blood collection tube which serves as a main component of the vacuum blood collector is mainly used for the collection and preservation of a blood sample. The vacuum blood collection tube is preset with a certain amount of negative pressure during the production process. When the blood collection needle punctures a blood vessel, the blood automatically flows into the blood collection tube due to the negative pressure in the blood collection tube. In order to ensure that medical staff are not stabbed by the used blood collection needle, a safety protective cover is also provided. The existing safety protective cover is a sleeve-type protective cover, which is difficult to align with a needle tip and easily injuries others accidentally. A retractable safety protective cover came into being. This kind of safety protective cover is usually arranged outside the blood collection needle in a sleeving manner. When the blood collection needle is in use, it moves along a slideway on the side wall of the protective cover to a needle tip through a fin provided on the needle seat side until the blood collection needle is pushed to a proper position and limited, and then the blood collection operation is performed. After the operation is completed, the blood collection tube retracts into the safety protective cover, thereby achieving the purpose of safety protection. However, there are several problems for the retractable safety protection device on the market: 1. a contact of the push-type retractable protection device protrudes from the outside of a sleeve, and is thus easily touched accidentally; and 2, the push-type retractable protection device is operated in need of the cooperation of both hands, rather than a left hand or a right hand alone, which brings inconvenience to the operations of the medical staff.

### SUMMARY

An object of the present invention is to provide a push-type safety protection mechanism of a blood collection needle, so as to solve the above-mentioned problems of the prior art.

The object of the present invention is achieved by the following technical solution.

A push-type safety protection mechanism of a blood collection needle comprises a sleeve member and a valve core member which is accommodated in the sleeve member and slides forward and backward inside the sleeve member, wherein the valve core member is a hollow pipe and provided with three parts, namely a needle seat part which is positioned at the front part and connected to the blood collection needle, a sleeving part which is positioned at the rear part and connected to a guide pipe, and a stopping part which is at least partly higher than the needle seat part; a plurality of limiting grooves is formed in the stopping part; the outside of the needle seat part is sleeved with a compression spring, wherein one end of the compression spring abuts against the stopping part, and the other end of the compression spring abuts against the inner side wall of a mouth of the sleeve member; the sleeve member is provided with two parts, namely a first sleeve part positioned at a front section and a second sleeve part positioned at a rear section; an elastic button higher than the side wall of the first sleeve part is arranged on the side wall of the valve core member; a limiting opening matched with the elastic button is formed in the side wall of the first sleeve part; a limiting sliding cavity for accommodating the elastic button and the valve core member is formed from a position of the first sleeve part from the limiting opening to the inner side of the second sleeve part, and the limiting sliding cavity guides and limits front-back movements of the elastic button and the valve core member; and a plurality of elastic limiting protrusions matched with the limiting grooves is arranged on the inner wall of the tail sliding cavity of the second sleeve part.

The axis of the sliding cavity can be set to be inclined to the opposite side of the elastic button. In this way, when the blood collection needle is pushed out, a needle tip is inclined toward the human body, which is convenient for medical staff to collect blood.

The first sleeve part and the second sleeve part are connected by one or more structures of sleeving, bonding, clamping and welding.

The elastic button extends from the sleeving part to the needle seat part, and forms a certain included angle with the valve core member; and a step part which is mutually buckled with the limiting opening is arranged at the end of the elastic button.

When the valve core member and the elastic button retract in the limiting sliding cavity, the valve core member is inclined toward one side under the joint action of the valve core member, the elastic button and the limiting sliding cavity; and when the blood collection needle completely enters the sleeve member, an inclined surface of a needle tip of the blood collection needle contacts the inner wall of the sleeve member. This will prevent blood from splashing out.

More specifically, in the case of sleeving connection, the first sleeve part is arranged outside the second sleeve part in a sleeving manner, and an overlapping part of the first sleeve part and the second sleeve part has a space matched with the limiting sliding cavity.

The plurality of limiting grooves is mutually continuous to form a limiting groove that surrounds the valve core member.

A fin for a blood collection operation is arranged on the side wall of the first sleeve part. In addition, the wing may be arranged on one side or both sides of the first sleeve part, which is convenient for medical staff with different habits of using the left hand or the right hand. The surface of the fin is provided with a plurality of salient points to increase the friction.

In order to facilitate the blood collection operation, the fin is located in the front of the center of gravity of the protection mechanism, so that it is easy to exert force during use without shaking.

A plurality of protruding ribs is arranged on the outer side wall of the second sleeve part, which increases the strength on the one hand, and increases the friction on the other hand, so as to facilitate the one-handed blood collection operation.

The first sleeve part is also matched with a safety protective cap which is arranged outside the elastic button in a sleeving manner. More specifically, the mouth of the safety protection cap abuts against the second sleeve part.

The tail end surface of the second sleeve part is provided with an inwardly-curled edge to restrict the valve core member from falling off.

The side wall of the sleeve member is provided with at least one transparent area for observation. Specifically, the transparent area may be a transparent observation window, or the entire sleeve member is made of a transparent material.

The “front” in the present invention refers to a direction of the needle tip of the blood collection needle; and the “rear” or “tail” refers to a direction away from the needle tip.

The present invention has the following beneficial effects:

1, through the design of the elastic button, medical staff can operate with the left hand or the right hand alone during use;

2, by arranging the sleeve member into two parts with different heights, an operation area including the elastic button and the limiting opening is located near the second sleeve part, which increases the difficulty in an abnormal contact between the elastic button and the human body and increases the reliability;

3, the elastic button slides in the limiting sliding cavity in the second sleeve part during the entire retraction process, which can prevent a misoperation to the greatest extent; and

4, through the special safety protective cap, the elastic button device is protected in the safety protective cap, thereby avoiding the retraction of the blood collection needle caused by the misoperation.

### BRIEF DESCRIPTION OF THE DRAWINGS

FIG. 1 is a schematic structural diagram in the installation of the present invention;

FIG. 2 is a schematic structural diagram after a valve core member and a sleeve member of the present invention are assembled (not including a blood collection needle and a safety protective cap); and

FIG. 3 is a schematic structural diagram of the valve core member of the present invention.

In drawings, the reference symbols represent the following components: 10—sleeve member; 11—first sleeve part; 12—second sleeve part; 13—limiting opening; 14—fin; 15—protruding rib; 16—elastic limiting protrusion; 17—limiting sliding cavity; 20—spring; 21—valve core member; 31—needle seat part; 32—stopping part; 33—sleeving part; 34—limiting groove; 35—elastic button; 40—safety protective cap.

### DETAILED DESCRIPTION

The structural features of the present invention are further described below in conjunction with the accompanying drawings and specific embodiments.

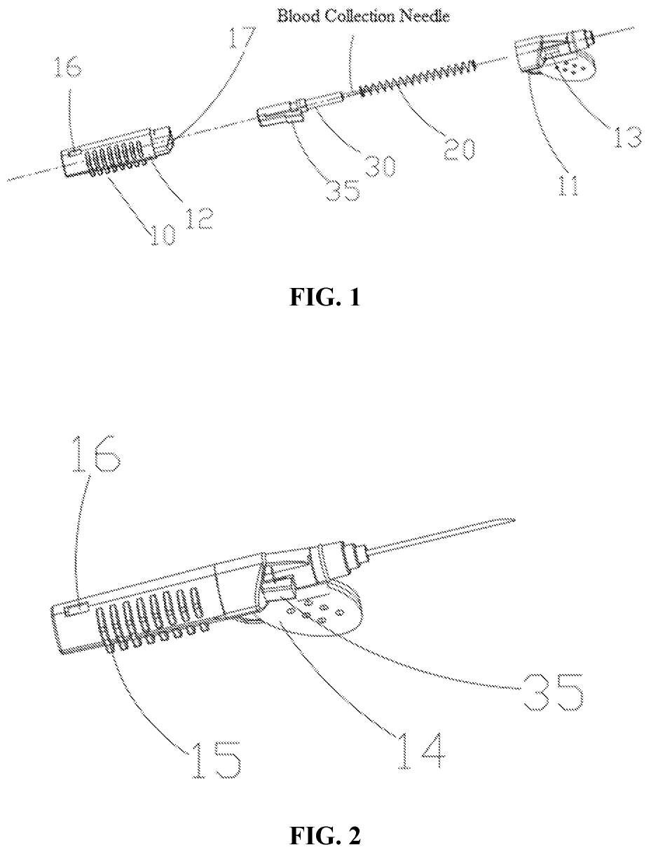

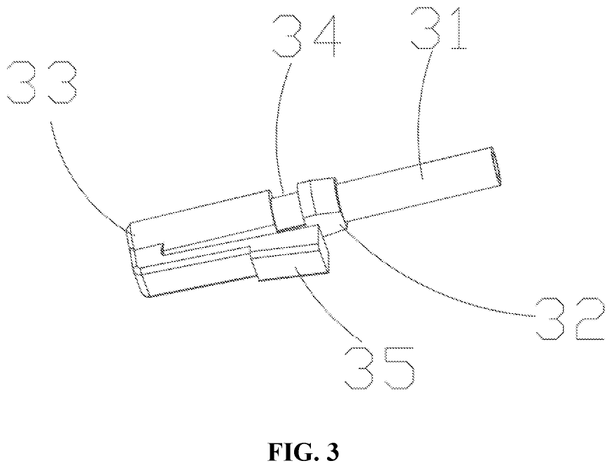

A push-type safety protection mechanism of a blood collection needle as shown in FIG. 1 to FIG. 3 comprises a sleeve member 10 and a valve core member 30 which is accommodated in the sleeve member 10 and slides forward and backward inside the sleeve member, wherein the valve core member 30 is a hollow pipe and provided with three parts, namely a needle seat part 31 which is positioned at the front part and connected to the blood collection needle, a sleeving part 33 which is positioned at the rear part and connected to a guide pipe, and a stopping part which is at least partly higher than the needle seat part 31; a plurality of limiting grooves 34 is formed in the stopping part; the outside of the needle seat part 31 is sleeved with a compression spring 20, wherein one end of the compression spring 20 abuts against the stopping part 32, and the other end of the compression spring 20 abuts against the inner side wall of a mouth of the sleeve member 10; the sleeve member 10 is provided with two parts, namely a first sleeve part 11 positioned at a front section and a second sleeve part 12 positioned at a rear section; an elastic button higher than the side wall of the first sleeve part is arranged on the side wall of the valve core member 30; a limiting opening matched with the elastic button is formed in the side wall of the first sleeve part; a limiting sliding cavity for accommodating the elastic button and the valve core member is formed from a position of the first sleeve part from the limiting opening to the inner side of the second sleeve part, and the limiting sliding cavity guides and limits front-back movements of the elastic button 35 and the valve core member 30; and a plurality of elastic limiting protrusions matched with the limiting grooves 34 is arranged on the inner wall of the tail sliding cavity 17 of the second sleeve part 12.

The axis of the sliding cavity can be set to be inclined to the opposite side of the elastic button. In this way, when the blood collection needle is pushed out, a needle tip is inclined toward the human body, which is convenient for medical staff to collect blood.

The first sleeve part 11 and the second sleeve part 12 are connected by one or more structures of sleeving, bonding, clamping and welding.

The elastic button extends from the sleeving part to the needle seat part, and forms a certain included angle with the valve core member; and a step part which is mutually buckled with the limiting opening is arranged at the end of the elastic button.

More specifically, in the case of sleeving connection, the first sleeve part 11 is arranged outside the second sleeve part 12 in a sleeving manner, and an overlapping part of the first sleeve part 11 and the second sleeve part 12 has a space matched with the limiting sliding cavity 17.

The plurality of limiting grooves 34 is mutually continuous to form a limiting groove 34 that surrounds the valve core member.

A fin 14 for a blood collection operation is arranged on the side wall of the first sleeve part 11. The fin 14 may be arranged on one side or both sides of the first sleeve part, which is convenient for medical staff with different habits of using the left hand or the right hand. The surface of the fin 14 is provided with a plurality of salient points to increase the friction.

In order to facilitate the blood collection operation, the fin is located in the front of the center of gravity of the protection mechanism, so that it is easy to exert force during use without shaking.

A plurality of protruding ribs 15 is arranged on the outer side wall of the second sleeve part 12, which increases the strength on the one hand, and increases the friction on the other hand, so as to facilitate the one-handed blood collection operation.

The first sleeve part 12 is also matched with a safety protective cap 40 which is arranged outside the elastic button in a sleeving manner. More specifically, the mouth of the safety protection cap 40 abuts against the second sleeve part 12, which can protect the needle tip on the one hand, and protect the elastic button from being touched accidentally on the other hand.

The tail end surface of the second sleeve part 12 is provided with an inwardly-curled edge to restrict the valve core member 30 from falling off.

During installation, the safety protective cap is arranged on the first sleeve part in a sleeving manner. The blood collection needle and the guide pipe are installed to the needle seat part and the sleeving part of the valve core member, respectively. The spring is arranged outside the needle seat part in a sleeving manner and together put into the first sleeve part together, and the elastic button is pushed to the limiting opening until the step at the end of the elastic button is buckled with the limiting opening. The second sleeve part is fixed with the first sleeve part.

After the blood collection operation is completed, the elastic button is pushed, and the step part of the elastic button enters the sleeve member under the action of the external force. When the valve core member and the elastic button retract in the limiting sliding cavity, the valve core member is inclined toward one side under the joint action of the valve core member, the elastic button and the limiting sliding cavity; and when the blood collection needle completely enters the sleeve member, an inclined surface of a needle tip of the blood collection needle contacts the inner wall of the sleeve member. This will prevent blood from splashing out. At this time, the valve core member retracts into the second sleeve part under the action of the spring, and the limiting grooves and the elastic limiting protrusions are engaged.

What is claimed is:

1. A push-type safety protection mechanism of a blood collection needle, comprising a sleeve member and a valve core member which is accommodated in the sleeve member and slides forward and backward inside the sleeve member, wherein the valve core member is a hollow pipe and provided with three parts, namely a needle seat part which is positioned at the front part and connected to the blood collection needle, a sleeving part which is positioned at the rear part and connected to a guide pipe, and a stopping part which is at least partly higher than the needle seat part; a plurality of limiting grooves is formed in the stopping part; the outside of the needle seat part is sleeved with a compression spring, wherein one end of the compression spring abuts against the stopping part, and the other end of the compression spring abuts against the inner side wall of a mouth of the sleeve member; the sleeve member is provided with two parts, namely a first sleeve part positioned at a front section and a second sleeve part positioned at a rear section; an elastic button higher than the side wall of the first sleeve part is arranged on the side wall of the valve core member; a limiting opening matched with the elastic button is formed in the side wall of the first sleeve part; a limiting sliding cavity for accommodating the elastic button and the valve core member is formed from a position of the first sleeve part from the limiting opening to the inner side of the second sleeve part, and the limiting sliding cavity guides and limits front-back movements of the elastic button and the valve core member; and a plurality of elastic limiting protrusions matched with the limiting grooves is arranged on the inner wall of the tail sliding cavity of the second sleeve part.

2. The push-type safety protection mechanism of the blood collection needle according to claim 1, wherein the axis of the sliding cavity can be set to be inclined to the opposite side of the elastic button.

3. The push-type safety protection mechanism of the blood collection needle according to claim 1, wherein the first sleeve part and the second sleeve part are connected by one or more structures of sleeving, bonding, clamping and welding.

4. The push-type safety protection mechanism of the blood collection needle according to claim 1, wherein the elastic button extends from the sleeving part to the needle seat part, and forms a certain included angle with the valve core member; and a step part which is mutually buckled with the limiting opening is arranged at the end of the elastic button.

5. The push-type safety protection mechanism of the blood collection needle according to claim 1, wherein when the valve core member and the elastic button retract in the limiting sliding cavity, the valve core member is inclined toward one side under the joint action of the valve core member, the elastic button and the limiting sliding cavity; and when the blood collection needle completely enters the sleeve member, an inclined surface of a needle tip of the blood collection needle contacts the inner wall of the sleeve member.

6. The push-type safety protection mechanism of the blood collection needle according to claim 1, wherein the plurality of limiting grooves is mutually continuous to form a limiting groove that surrounds the valve core member.

7. The push-type safety protection mechanism of the blood collection needle according to claim 1, wherein a fin for a blood collection operation is arranged at one side or both sides of the side wall of the first sleeve part.

8. The push-type safety protection mechanism of the blood collection needle according to claim 7, wherein the surface of the fin is provided with a plurality of salient points.

9. The push-type safety protection mechanism of the blood collection needle according to claim 1, wherein a plurality of protruding ribs is arranged on the outer side wall of the second sleeve part; and the tail end surface of the second sleeve part is provided with an inwardly-curled edge to restrict the valve core member from falling off.

10. The push-type safety protection mechanism of the blood collection needle according to claim 1, wherein the first sleeve part is also matched with a safety protective cap which is arranged outside the elastic button in a sleeving manner.

11. The push-type safety protection mechanism of the blood collection needle according to claim 10, wherein the mouth of the safety protective cap abuts against the second sleeve part.

12. The push-type safety protection mechanism of the blood collection needle according to claim 1, wherein the side wall of the sleeve member is provided with at least one transparent area for observation.
